SEQUENCE LISTING (2 sequences)

SEQ ID NO: 1 - DNA, 56 bp, Artificial
  Synthetic ShRNA-RB1 forward primer
accgcagtactaagggaacaataagttctctattgttcccttagtactgctttttc

SEQ ID NO: 2 - DNA, 56 bp, Artificial
  Synthetic ShRNA-RB1 reverse primer
tgcagaaaaagcagtactaagggaacaatagagaacttattgttcccttagtactg

(19) United States
(12) Patent Application Publication
(10) Pub. No.: US 2013/0195863 A1
(43) Pub. Date: Aug. 1, 2013

(54) Methods and Pharmaceutical Compositions for the Treatment of Bone Density Related Diseases

(71) Applicants: Philippe Clezardin, Lyon (FR); Vincent Gonin, Lyon (FR); Richard Bachelier, Lyon (FR); Edith Bonnelye, Lyon (FR)

(72) Inventors: Philippe Clezardin, Lyon (FR); Vincent Gonin, Lyon (FR); Richard Bachelier, Lyon (FR); Edith Bonnelye, Lyon (FR)

(21) Appl. No.: 13/823,907

(22) PCT Filed: Sep. 28, 2010
(86) PCT No.: PCT/IB2010/002632

§ 371 Date: Mar. 15, 2013

(51) Int. Cl. A61K 39/395; A61K 31/7105; A61K 38/17; A61K 48/00; G01N 33/68; C12Q 1/68

(52) U.S. Cl. CPC A61K 39/3955; G01N 33/6893; C12Q 1/6876; A61K 38/1709; A61K 48/005; A61K 31/7105
USPC 424/134.1; 435/7.21; 435/6.12; 436/501; 424/172.1; 514/16.7; 514/44 R; 514/44 A

(57) ABSTRACT

The invention relates to methods and pharmaceutical compositions for the treatment of bone density related diseases. More particularly, the present invention relates to a ROBO1 modulator for use in a method for the treatment of a bone mineral density related disease in a subject. In a particular embodiment the ROBO1 modulator is selected from the group consisting of small organic molecules, antibodies, aptamers or polypeptides. In another particular embodiment said bone mineral density related disease is selected from the group consisting of ghosal hematodiaphyseal dysplasia syndrome (GHDD), osteoporosis, osteoporosis associated to pseudoglioma, osteoporosis and oculocutaneous hypopigmentation syndrome, osteoporosis due to endocrinological dysfunction, osteogenesis imperfecta osteopenia, Paget's disease, osteomyelitis, hypercalcemia, osteonecrosis, hyperparathyroidism, lytic bone metastases, periodontitis, bone loss due to immobilization and osteoporosis associated with a disease selected from the group consisting of cachexia, anorexia, alopecia, rheumatoid arthritis, psoriatic arthritis, psoriasis, and inflammatory bowel disease.

### FIELD OF THE INVENTION

The invention relates to methods and pharmaceutical compositions for the treatment of bone density related diseases.

### BACKGROUND OF THE INVENTION

Morphogenesis and remodeling of bone are accomplished by the coordinated actions of bone-resorbing osteoclasts and bone-forming osteoblasts, which metabolize and remodel bone structure throughout development and adult life. Bone is constantly being resorbed and formed at specific sites in the skeleton called basic multicellular units. An estimated 10% of the total bone mass in the human body is remodeled each year. Upon activation, osteoclasts, which differentiate from hematopoietic monocyte/macrophage precursors, migrate to the basic multicellular unit, resorb a portion of bone and finally undergo apoptosis. Subsequently, newly generated osteoblasts, arising from preosteoblastic/stromal cells, form bone at the site of resorption. The development of osteoclasts is controlled by preosteoblastic cells, so that the processes of bone resorption and formation are tightly coordinated, thus allowing for a wave of bone formation to follow each cycle of bone resorption.

Imbalances between osteoclast and osteoblast activities can result in skeletal abnormalities characterized by decreased (osteoporosis) or increased (osteopetrosis) bone density.

For example, osteoporosis, or porous bone, is a disease characterized by low bone mineral density, leading to bone fragility and an increased susceptibility to fractures, especially of the hip, spine and wrist, although any bone can be affected. If not prevented or if left untreated, osteoporosis can progress painlessly until a bone breaks. It is estimated that osteoporosis is responsible for more than 1.5 million fractures annually, including over 300,000 hip fractures; and approximately 700,000 vertebral fractures; 250,000 wrist fractures; and 300,000 fractures at other sites.

Bone metastases are a frequent complication of malignancy, occurring in the majority of patients with advanced breast and prostate cancer and multiple myeloma, as well as between 15-30% of patients with cancers of the lung, colon, stomach, bladder, uterus, thyroid and kidney. It is estimated that each year in the United States 350,000 people die with bone metastases. Bone metastases have been characterized as osteolytic and osteoblastic, both of which frequently cause intractable bone pain, pathological fractures, life-threatening hypercalcemia and various nerve compression syndromes.

Bisphosphonates are widely used to inhibit osteoclast activity in a variety of both benign and malignant diseases which involve excessive bone resorption. These pyrophosphate analogs not only reduce the occurrence of skeletal related events (e.g., fractures, need for radiation therapy, spinal cord compression, hypercalcemia of malignancy) but they also provide patients with further clinical benefit (e.g., pain reduction) and potentially improve survival. Bisphosphonates are able to prevent bone resorption in vivo; the therapeutic efficacy of bisphosphonates has been demonstrated in the treatment of osteoporosis, osteopenia, Paget's disease of bone, tumor-induced hypercalcemia (TIH) and, more recently, bone metastases (BM) from solid tumors and bone lesions from multiple myeloma (MM). The mechanisms by which bisphosphonates inhibit bone resorption are still not completely understood and seem to vary according to the bisphosphonates studied. Bisphosphonates have been shown to bind strongly to the hydroxyapatite crystals of bone, to reduce bone turnover and resorption, to decrease the levels of hydroxyproline or alkaline phosphatase in the blood, and in addition to inhibit the formation, recruitment, activation and the activity of osteoclasts.

Communication between osteoblasts and osteoclasts occurs through cytokines and cell-to-cell contacts. A cytokine that performs a key regulatory role in bone remodeling is receptor activator of NF-kappaB ligand (RANKL). Inappropriate activation of osteoclasts by RANKL can indeed create an imbalance between the processes of bone resorption, resulting in the rate of bone resorption exceeding that of bone formation. Therefore, anti-RANKL neutralizing antibodies have been suggested for the treatment of osteoporosis and bone metastases.

However most agents used to treat osteoporosis, such as estrogens and bisphosphonates, are not very effective. These agents retard bone resorption but do not improve connectivity. Therefore, there is an existing need to identify factors which impact the bone mineral density so as to envisage methods for diagnosing, predicting, preventing and treating bone mineral density related diseases.

### SUMMARY OF THE INVENTION

The invention relates to methods and pharmaceutical compositions for the treatment of bone density related diseases.

More particularly, the present invention relates to a ROBO1 modulator for use in a method for the treatment of a bone mineral density related disease in a subject. In a particular embodiment the ROBO1 modulator is selected from the group consisting of small organic molecules, antibodies, aptamers or polypeptides. In another particular embodiment said bone mineral density related disease is selected from the group consisting of ghosal hematodiaphyseal dysplasia syndrome (GHDD), osteoporosis, osteoporosis associated to pseudoglioma, osteoporosis and oculocutaneous hypopigmentation syndrome, osteoporosis due to endocrinological dysfunction, osteogenesis imperfecta osteopenia, Paget's disease, osteomyelitis, hypercalcemia, osteonecrosis, hyperparathyroidism, lytic bone metastases, periodontitis, bone loss due to immobilization and osteoporosis associated with a disease selected from the group consisting of cachexia, anorexia, alopecia, rheumatoid arthritis, psoriatic arthritis, psoriasis, and inflammatory bowel disease.

In a further aspect the present invention relates to a ROBO1 agonist for use in a method for the treatment of a bone mineral density related disease associated with a decreased bone mineral density. In a particular embodiment, the ROBO1 agonist is a SLIT2 polypeptide or a fusion protein comprising the following segments: a SLIT2 polypeptide and the Fc domain of an immunoglobulin or a function-conservative variant thereof. In another particular embodiment said bone mineral density related disease associated with a decreased bone mineral density is selected from the group consisting of osteoporosis, osteogenesis imperfecta osteopenia, Paget's disease, osteomyelitis, hypercalcemia, osteonecrosis, hyperparathyroidism, lytic bone metastases, and periodontitis.

A further aspect of the present invention relates to a ROBO1 antagonist for use in a method for the treatment of a bone mineral density related disease associated with an increased bone mineral density. In a particular embodiment, the ROBO1 antagonist according is selected from the group consisting of small organic molecules, antibodies, or aptamers. In another particular embodiment, the antibodies or aptamers are directed against ROBO1 or SLIT2. In another embodiment, said bone mineral density related disease associated with an increased bone mineral density is Ghosal hematodiaphyseal dysplasia syndrome (GHDD).

A further aspect of the invention relates to a nucleic acid molecule encoding for an SLIT2 polypeptide or a fusion protein comprising thereof for use in a method for the treatment of a bone mineral density related disease is associated with a decreased bone mineral density.

A further aspect of the invention relates to an inhibitor of ROBO1 or SLIT2 gene expression for use in a method for the treatment of a bone mineral density related disease associated with an increased bone mineral density

A further aspect of the invention relates to a pharmaceutical composition comprising a ROBO1 modulator of the invention for use in a method for the treatment of a bone mineral density related disease.

A further aspect of the invention relates to a biomaterial or medical delivery device for use in a method for the treatment of a bone mineral density related disease comprising an amount of a ROBO1 modulator.

A further aspect of the invention relates to a method of testing a patient thought to have or be predisposed to a bone density related disease, which comprises the step of analyzing a biological sample from said patient for:

a. detecting the presence of a mutation in the gene encoding for ROBO1 or SLIT2 and/or its associated promoter, and/or

b. analyzing the expression of the gene encoding for ROBO1 or SLIT2.

### DETAILED DESCRIPTION OF THE INVENTION

### Therapeutic Methods of the Invention

The present invention relates to a ROBO1 modulator for use in a method for the treatment of a bone mineral density related disease in a subject.

As used herein the term “ROBO1” has its general meaning in the art and denotes the cell surface transmembrane protein Roundabout 1. ROBO1 was first described as an axon guidance receptor protein, and its amino acid sequence and the gene sequence coding thereof are disclosed in GenBank ID NM_{—}002941 for variant 1, and GenBank ID NM_{—}133631 for variant 2. The natural ligand of ROBO1 is SLIT2.

As used herein, “SLIT2” refers to a member of the Slit family of “neurological” migratory cues. It is expressed by midline cells and endothelial cells and functions as a repellent in axon guidance and branching, neuronal migration, and as an endogenous inhibitor for leukocyte chemotaxis. Slit2 is a large multidomain protein containing an unusual domain organization of four tandem leucine-rich repeat (LRR) domains at its N-terminus. These domains are well known to mediate protein-protein interactions; indeed, the Robo1-binding region has been mapped to the concave face of the second LRR domain. Currently, there are three slit genes, slit 1, 2 and 3, known in the mammals. Its amino acid sequence and the gene sequence coding thereof are disclosed in GenBank ID NM_{—}004787. However, it should be understood that, as those of skill in the art are aware of the sequence of these molecules, any SLIT2 protein or gene sequence variant may be used as long as it has the properties of an SLIT2. Accordingly, the term encompasses the function conservative variants of SLIT2.

“Function conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, most preferably at least 85%, and even more preferably at least 90%, and which has the same or substantially similar properties or functions as the native or parent protein to which it is compared.

As used herein, the term “ROBO1 modulator” denotes a natural or synthetic compound which binds to the ROBO1 and which modulates (i.e. activates or inhibits) said receptor. The term modulator includes agonists and antagonists. As used herein, the term “ROBO1 agonist” is a natural or synthetic compound which binds the ROBO1 to activate said ROBO1 for initiating a pathway signalling and further biological processes. As used herein, the term “ROBO1 antagonist” denotes a natural or synthetic compound that has a biological effect opposite to that of an agonist.

The ROBO1 antagonistic or agonistic activity may be determined using various methods as described by Rhee et al. (Nat Cell Biol., 4:798-805, 2002), Wang et al. (Cancer Cell, 4:19-29, 2003), Marlow et al. (Cancer Res., 68:7819-7827, 2008), Stella et al. (Mol Biol Cell, 20:642-657, 2009) and Sheldon et al., (FASEB J., 23:513-522, 2009). Typically, the overexpression of SLIT2 or ectopic expression of a soluble decoy ROBO1 are strategies that are used to investigate functions of the ROBO1/SLIT2 system.

The term “bone mineral density related disease” refers to a condition which is characterized at least in part by an increase in bone resorption and/or a loss of bone mass or bone density. The term “bone mineral density related disease” includes, but is not limited to, ghosal hematodiaphyseal dysplasia syndrome (GHDD), osteoporosis, osteogenesis imperfecta osteopenia, Paget's disease, osteomyelitis, hypercalcemia, osteonecrosis, hyperparathyroidism, lytic bone metastases, periodontitis, and bone loss due to immobilization.

As used herein the term “osteoporosis” has its general meaning in the art and includes any form of osteoporosis. For example, osteoporosis includes primary osteoporosis, post-menopausal and age-related osteoporosis, endocrine osteoporosis (including hyperthyroidism, hyperparathyroidism, Gushing's syndrome, and acromegaly), hereditary and congenital forms of osteoporosis (including osteogenesis imperfecta, homocystinuria, Menkes' syndrome, Riley-Day syndrome), and osteoporosis due to immobilization of extremities. The term also includes osteoporosis that is secondary to other disorders, including hemochromatosis, hyperprolactinemia, anorexia nervosa, thyrotoxicosis, diabetes mellitus, celiac disease, inflammatory bowel disease, primary biliary cirrhosis, rheumatoid arthritis, ankylosing spondylitis, multiple myeloma, lymphoproliferative diseases, and systemic mastocytosis. The term also includes osteoporosis secondary to surgery (e.g., gastrectomy) or to drug therapy, including chemotherapy, endocrine therapy, anticonvulsant therapy, immunosuppressive therapy, and anticoagulant therapy. The term also includes osteoporosis secondary to glucocorticosteroid treatment for certain diseases, including rheumatoid arthritis (RA), systemic lupus erythematosus (SLE), asthma, temporal arthritis, vasculitis, chronic obstructive pulmonary disease, polymyalgia rheumatica, polymyositis, and chronic interstitial lung disease. The term also includes osteoporosis secondary to glucocorticosteroid and/or immunomodulatory treatment to prevent organ rejection following organ transplant such as kidney, liver, lung, and heart transplants. The term also includes osteoporosis due to submission to microgravity, such as observed during space travel. The term also includes osteoporosis associated with malignant disease, such as breast cancer, prostate cancer.

In the context of the invention, the term “treating” or “treatment”, as used herein, means preventing, reversing and alleviating, inhibiting the progress of the condition to which such term applies condition.

As used herein, the term “subject” denotes a mammal, such as a rodent, a feline, a canine, and a primate. Preferably, a subject according to the invention is a human.

In a particular embodiment, the present invention relates to a ROBO1 agonist for use in a method for the treatment of a bone mineral density related disease associated with a decreased bone mineral density. Examples of such diseases include but are not limited to osteoporosis, osteogenesis imperfecta osteopenia, Paget's disease, osteomyelitis, hypercalcemia, osteonecrosis, hyperparathyroidism, lytic bone metastases, and periodontitis.

According to the invention the bone metastasis results from cancer elsewhere in the body, for example, prostate cancer, breast cancer, lung cancer, melanoma, pancreatic cancer, colorectal cancer, ovarian cancer and brain cancer. In particular, the ROBO1 agonist according to the invention is for treating, preventing or alleviating the symptoms of bone metastasis, more particularly in the treatment of bone metastasis in patients with cancer, even more particularly in the treatment of patients with breast and prostate cancer. The ROBO1 agonist according to the invention may also be used to prevent or alleviate symptoms of pain associated with bone metastases and risk of pathological fractures and hypercalcemia.

In a particular embodiment, the present invention relates to a ROBO1 antagonist for use in a method for the treatment of a bone mineral density related disease associated with an increased bone mineral density. Examples of such diseases include but are not limited to Ghosal hematodiaphyseal dysplasia syndrome (GHDD).

In a particular embodiment, the ROBO1 modulator according to the invention is a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

Alternatively, the ROBO1 modulator (e.g. agonist or antagonist) may consist in an antibody (the term including “antibody fragment”). In particular, the ROBO1 modulator may consist in an antibody directed against the ROBO1 or its natural ligand SLIT2, in such a way that said antibody modulates ROBO1.

Antibodies can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies can be prepared and isolated using any technique that provides for the production of antibody molecules by continuous cell lines in culture. Techniques for production and isolation include but are not limited to the hybridoma technique; the human B-cell hybridoma technique; and the EBV-hybridoma technique. Alternatively, techniques described for the production of single chain antibodies (see, e.g., U.S. Pat. No. 4,946,778) can be adapted to produce anti-ROBO1 (or anti-SLIT2) single chain antibodies. The ROBO1 modulator (e.g. agonist or antagonist) useful in practicing the present invention also include anti-ROBO1 (or anti-SLIT2) antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to ROBO1 (or SLIT2).

Humanized antibodies and antibody fragments thereof can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then after raising antibodies as above described, the skilled man in the art can easily select those modulating ROBO1.

In another embodiment the ROBO1 modulator is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods.

Then after raising aptamers directed against the ROBO1 (or SLIT2) as above described, the skilled man in the art can easily select those modulating ROBO1.

In another particular embodiment, the ROBO1 modulator according to the invention may consist in a polypeptide.

More particularly, a ROBO1 agonist according to the invention may consist in a SLIT2 polypeptide.

According to the invention the term “SLIT2 polypeptide” refers to any polypeptide that consist in SLIT2 or a fragment thereof provided that said fragment is capable to bind and activate ROBO1. For example, it refers to fragments encompassing the second leucine-reach repeat domain of SLIT2, which specifically interacts with ROBO1.

In another particular embodiment, the ROBO1 agonist according to the invention may consist in a fusion protein comprising the following segments:

(a) a SLIT2 polypeptide as above described and

(b) the Fc domain of an immunoglobulin or a function-conservative variant thereof.

Segment (b) of said fusion protein serves at least one of the following purposes: secretion of the fusion protein from cells that produce said fusion protein, providing segment (a) in a form (e.g. folding or aggregation state) functional for binding ROBO1, affinity purification of said fusion protein, recognition of the fusion protein by an antibody, providing favourable properties to the fusion protein when used as a medicament. Surprisingly and most importantly, segment (b) allows production of said fusion protein in mammalian, preferably human, cells and secretion to the cell supernatant in active form, i e in a form functional for binding to ROBO1. Segment (b) may be antibody-derived, for example it may comprise an amino acid sequence of an immunoglobulin heavy chain constant part, and is most preferably an Fc domain of an immunoglobulin. Suitable immunoglobins are IgG, IgM, IgA, IgD, and IgE. IgG and IgA are preferred IgGs are most preferred, e.g. an IgG1. Said Fc domain may be a complete Fc domain or a function-conservative variant thereof. A variant of Fc is function-conservative if it retains at least one of the functions of segment (b) listed above. Segments (a) and (b) of the fusion protein of the invention may be linked by a linker. The linker may consist of about 1 to 100, preferably 1 to 10 amino acid residues.

In specific embodiments, it is contemplated that polypeptides of the present invention (e.g. SLIT2 polypeptides or fusion proteins comprising thereof) used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 45 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes (see e.g., technologies of established by VectraMed, Plainsboro, N.J.). Such linkers may be used in modifying the polypeptides (e.g. SLIT2 polypeptides or fusion proteins comprising thereof) described herein for therapeutic delivery.

According to the invention, the polypeptides according to the present invention (e.g. SLIT2 polypeptides or fusion proteins comprising thereof) may be produced by conventional automated peptide synthesis methods or by recombinant expression. General principles for designing and making proteins are well known to those of skill in the art.

Polypeptides of the invention (e.g. SLIT2 polypeptides or fusion proteins comprising thereof) may be synthesized in solution or on a solid support in accordance with conventional techniques. Various automatic synthesizers are commercially available and can be used in accordance with known protocols. polypeptides of the invention (e.g. SLIT2 polypeptides or fusion proteins comprising thereof) may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art.

As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Those of skill in the art are aware of various techniques for optimizing mammalian expression of proteins. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the recombinant production of the polypeptides of the invention (e.g. SLIT2 polypeptides or fusion proteins comprising thereof), it would be necessary to employ vectors comprising polynucleotide molecules for encoding the polypeptides of the invention (e.g. SLIT2 polypeptides or fusion proteins comprising thereof). Methods of preparing such vectors as well as producing host cells transformed with such vectors are well known to those skilled in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect genes. Examples of regulatory sequences include transcriptional promoters, operators, or enhancers, mRNA ribosomal binding sites, and appropriate sequences which control transcription and translation.

The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the peptides or polypeptides of the invention will of course depend upon the specific host cell to be used, and is within the skill of the ordinary artisan. Methods for the construction of mammalian expression vectors are disclosed, for example, in EP-A-0367566; and WO 91/18982.

In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are well known in the art.

Preferred viruses for certain applications are the adenoviruses and adeno-associated (AAV) viruses, which are double-stranded DNA viruses that have already been approved for human use in gene therapy. Actually 12 different AAV serotypes (AAV1 to 12) are known, each with different tissue tropisms. Recombinant AAV are derived from the dependent parvovirus AAV2. The adeno-associated virus type 1 to 12 can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the protein of interest. Thus, a promoter nucleotide sequence is operably linked to a given DNA sequence if the promoter nucleotide sequence directs the transcription of the sequence.

Similarly, the phrase “under transcriptional control” means that the promoter is in the correct location and orientation in relation to the nucleic acid to control RNA polymerase initiation and expression of the gene. Any promoter that will drive the expression of the nucleic acid may be used. The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. Common promoters include, e.g., the human cytomegalovirus (CMV) immediate early gene promoter, the SV40 early promoter, the Rous sarcoma virus long terminal repeat, [beta]-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient to produce a recoverable yield of protein of interest. By employing a promoter with well known properties, the level and pattern of expression of the protein of interest following transfection or transformation can be optimized. Inducible promoters also may be used.

Another regulatory element that is used in protein expression is an enhancer. These are genetic elements that increase transcription from a promoter located at a distant position on the same molecule of DNA. Where an expression construct employs a cDNA insert, one will typically desire to include a polyadenylation signal sequence to effect proper polyadenylation of the gene transcript. Any polyadenylation signal sequence recognized by cells of the selected transgenic animal species is suitable for the practice of the invention, such as human or bovine growth hormone and SV40 polyadenylation signals.

Another aspect of the invention relates to a nucleic acid molecule encoding for an SLIT2 polypeptide (or a fusion protein comprising thereof) for use in a method for the treatment of a bone mineral density related disease is associated with a decreased bone mineral density.

Typically, said nucleic acid is a DNA or RNA molecule, which may be included in any suitable vector, such as a plasmid, cosmid, episome, artificial chromosome, phage or a viral vector as above described.

So, a further object of the invention relates to a vector comprising a nucleic acid encoding for an SLIT2 polypeptide (or a fusion protein comprising thereof) for use in a method for the treatment of a bone mineral density related disease is associated with a decreased bone mineral density.

A further object of the invention relates to a host cell comprising a nucleic acid as above described for use in a method for the treatment of a bone mineral density related disease is associated with a decreased bone mineral density.

A further object of the invention relates to an inhibitor of ROBO1 or SLIT2 gene expression for use in a method for the treatment of a bone mineral density related disease associated with an increased bone mineral density.

Inhibitors of expression for use in the present invention may be based on anti-sense oligonucleotide constructs. Anti-sense oligonucleotides, including anti-sense RNA molecules and anti-sense DNA molecules, would act to directly block the translation of ROBO1 or SLIT2 mRNA by binding thereto and thus preventing protein translation or increasing mRNA degradation, thus decreasing the level of ROBO1 or SLIT2, and thus activity, in a cell. For example, antisense oligonucleotides of at least about 15 bases and complementary to unique regions of the mRNA transcript sequence encoding ROBO1 or SLIT2 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of expression for use in the present invention. ROBO1 or SLIT2 gene expression can be reduced by contacting a subject or cell with a small double stranded RNA (dsRNA), or a vector or construct causing the production of a small double stranded RNA, such that ROBO1 or SLIT2 gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). All or part of the phosphodiester bonds of the siRNAs of the invention are advantageously protected. This protection is generally implemented via the chemical route using methods that are known by art. The phosphodiester bonds can be protected, for example, by a thiol or amine functional group or by a phenyl group. The 5′- and/or 3′-ends of the siRNAs of the invention are also advantageously protected, for example, using the technique described above for protecting the phosphodiester bonds. The siRNAs sequences advantageously comprises at least twelve contiguous dinucleotides or their derivatives.

As used herein, the term “siRNA derivatives” with respect to the present nucleic acid sequences refers to a nucleic acid having a percentage of identity of at least 90% with erythropoietin or fragment thereof, preferably of at least 95%, as an example of at least 98%, and more preferably of at least 98%.

As used herein, “percentage of identity” between two nucleic acid sequences, means the percentage of identical nucleic acid, between the two sequences to be compared, obtained with the best alignment of said sequences, this percentage being purely statistical and the differences between these two sequences being randomly spread over the nucleic acid acids sequences. As used herein, “best alignment” or “optimal alignment”, means the alignment for which the determined percentage of identity (see below) is the highest. Sequences comparison between two nucleic acids sequences are usually realized by comparing these sequences that have been previously align according to the best alignment; this comparison is realized on segments of comparison in order to identify and compared the local regions of similarity. The best sequences alignment to perform comparison can be realized, beside by a manual way, by using the global homology algorithm developed by SMITH and WATERMAN (Ad. App. Math., vol. 2, p:482, 1981), by using the local homology algorithm developed by NEDDLEMAN and WUNSCH (J. Mol. Biol., vol. 48, p:443, 1970), by using the method of similarities developed by PEARSON and LIPMAN (Proc. Natl. Acd. Sci. USA, vol. 85, p:2444, 1988), by using computer software using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C., Nucleic Acids Research, vol. 32, p:1792, 2004). To get the best local alignment, one can preferably used BLAST software. The identity percentage between two sequences of nucleic acids is determined by comparing these two sequences optimally aligned, the nucleic acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

shRNAs (short hairpin RNA) can also function as inhibitors of expression for use in the present invention.

Ribozymes can also function as inhibitors of expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of ROBO1 or SLIT2 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs, shRNAs and ribozymes of the invention may be delivered in vivo alone or in association with a vector as above described.

The present invention relates to a method for the treatment of a bone mineral density related disease in a subject in need thereof comprising the step of consisting of administrating said patient with therapeutically effective amount of a ROBO1 modulator according to the invention.

By a “therapeutically effective amount” is meant a sufficient amount of a ROBO1 modulator to treat a bone mineral density related disease at a reasonable benefit/risk ratio applicable to any medical treatment. It will be understood that the total usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved.

### Pharmaceutical Compositions According to the Invention

A further object of the invention relates to pharmaceutical compositions comprising a ROBO1 modulator of the invention for use in a method for the treatment of a bone mineral density related disease.

Typically, the ROBO1 modulator may be combined with pharmaceutically acceptable excipients, and optionally sustained-release matrices, such as biodegradable polymers, to form therapeutic compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The ROBO1 modulator can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the patient being treated. The person responsible for administration will, in any event, determine the appropriate dose for the individual patient.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

### Medical Devices According to the Invention

The present invention also relates to the use of an ROBO1 modulator of the present invention for the preparation of biomaterials or medical delivery devices selected among prostheses, internal patches around bone, or bone implants.

In this respect, the invention relates more particularly to biomaterials or medical delivery devices as mentioned above, comprising (e.g. coated with) an amount of a ROBO1 modulator as defined above. Such a local biomaterial or medical delivery device can be used to modulate bone density.

The compounds used for the coating of the prostheses should preferentially permit a controlled release of said inhibitor. Said compounds could be polymers (such as sutures, polycarbonate, Hydron, and Elvax), biopolymers/biomatrices (such as alginate, fucans, collagen-based matrices, heparan sulfate) or synthetic compounds such as synthetic heparan sulfate-like molecules or combinations thereof. Other examples of polymeric materials may include biocompatible degradable materials, e.g. lactone-based polyesters orcopolyesters, e.g. polylactide; polylactide-glycolide; polycaprolactone-glycolide; polyorthoesters; polyanhydrides; polyaminoacids; polysaccharides; polyphospha-zenes; poly (ether-ester) copolymers, e.g. PEO-PLLA, or mixtures thereof and biocompatible non-degrading materials, e.g. polydimethylsiloxane; poly (ethylene-vinylacetate); acrylate based polymers or copolymers, e.g. polybutylmethacrylate, poly (hydroxyethyl methyl-methacrylate); polyvinyl pyrrolidinone; fluorinated polymers such as polytetrafluoethylene; cellulose esters. When a polymeric matrix is used, it may comprise 2 layers, e.g. a base layer in which said ROBO1 modulator is incorporated, such as ethylene-co-vinylacetate and polybutylmethacrylate, and a top coat, such as polybutylmethacrylate, which acts as a diffusion-control of said ROBO1 modulator. Alternatively, said ROBO1 modulator may be comprised in the base layer and the adjunct may be incorporated in the outlayer, or vice versa.

Such biomaterial or medical delivery device may be biodegradable or may be made of metal or alloy, e.g. Ni and Ti, or another stable substance when intented for permanent use. The ROBO1 modulator may also be entrapped into the metal of graft body which has been modified to contain micropores or channels. Also internal patches made of polymer or other biocompatible materials as disclosed above that contain the ROBO1 modulator may also be used for local delivery.

Said biomaterial or medical delivery device allow the ROBO1 modulator releasing from said biomaterial or medical delivery device over time and entering the surrounding tissue. Said releasing may occur during 1 month to 1 year. The local delivery according to the present invention allows for high concentration of the ROBO1 modulator at the disease site with low concentration of circulating compound. The amount of said ROBO1 modulator used for such local delivery applications will vary depending on the compounds used, the condition to be treated and the desired effect. For purposes of the invention, a therapeutically effective amount will be administered.

The local administration of said biomaterial or medical delivery device preferably takes place at or near the bone lesions sites.

### Diagnostic Methods of the Invention

A further aspect of the invention relates to a method of testing a patient thought to have or be predisposed to a bone density related disease, which comprises the step of analyzing a biological sample from said patient for:

(i) detecting the presence of a mutation in the gene encoding for ROBO1 or SLIT2 and/or its associated promoter, and/or

(ii) analyzing the expression of the gene encoding for ROBO1 or SLIT2.

As used herein, the term “biological sample” refers to any sample from a patient such as blood or serum or even a bone sample or a tumour sample.

Typical techniques for detecting a mutation in the gene encoding for ROBO1 or SLIT2 may include restriction fragment length polymorphism, hybridisation techniques, DNA sequencing, exonuclease resistance, microsequencing, solid phase extension using ddNTPs, extension in solution using ddNTPs, oligonucleotide assays, methods for detecting single nucleotide polymorphism such as dynamic allele-specific hybridisation, ligation chain reaction, mini-sequencing, DNA “chips”, allele-specific oligonucleotide hybridisation with single or dual-labelled probes merged with PCR or with molecular beacons, and others.

Analyzing the expression of the gene encoding for ROBO1 or SLIT2 may be assessed by any of a wide variety of well-known methods for detecting expression of a transcribed nucleic acid or translated protein.

In a preferred embodiment, the expression of the gene encoding for ROBO1 or SLIT2 is assessed by analyzing the expression of mRNA transcript or mRNA precursors, such as nascent RNA, of said gene. Said analysis can be assessed by preparing mRNA/cDNA from cells in a biological sample from a patient, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip™ DNA Arrays (AFF YMETRIX).

Advantageously, the analysis of the expression level of mRNA transcribed from the gene encoding for ROBO1 or SLIT2 involves the process of nucleic acid amplification, e.g., by RT-PCR (the experimental embodiment set forth in U.S. Pat. No. 4,683,202), ligase chain reaction (BARANY, Proc. Natl. Acad. Sci. USA, vol. 88, p: 189-193, 1991), self sustained sequence replication (GUATELLI et al., Proc. Natl. Acad. Sci. USA, vol. 57, p: 1874-1878, 1990), transcriptional amplification system (KWOH et al., 1989, Proc. Natl. Acad. Sci. USA, vol. 86, p: 1173-1177, 1989), Q-Beta Replicase (LIZARDI et al., Biol. Technology, vol. 6, p: 1197, 1988), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In another preferred embodiment, the expression of the gene encoding for ROBO1 or SLIT2 is assessed by analyzing the expression of the protein translated from said gene. Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for ROBO1 or SLIT2.

Said analysis can be assessed by a variety of techniques well known from one of skill in the art including, but not limited to, enzyme immunoassay (EIA), radioimmunoassay (RIA), Western blot analysis and enzyme linked immunoabsorbant assay (RIA).

The method of the invention may comprise comparing the level of expression of the gene encoding for ROBO1 or SLIT2 in a biological sample from a patient with the normal expression level of said gene in a control. For example a significantly weaker level of expression of said gene in the biological sample of a patient as compared to the normal expression level is an indication that the patient has or is predisposed to developing a bone density related disease associated with decreased bone density. On the opposite a significantly higher level of expression of said gene in the biological sample of a patient as compared to the normal expression level is an indication that the patient has or is predisposed to developing a bone density related disease associated with an increased bone density. The “normal” level of expression of the gene encoding for ROBO1 or SLIT2 is the level of expression of said gene in a biological sample of a patient not afflicted with a bone density related disease. Preferably, said normal level of expression is assessed in a control sample (e.g., sample from a healthy patient, which is not afflicted by a bone density related disease) and preferably, the average expression level of said gene in several control samples.

The invention will be further illustrated by the following figures and examples. However, these examples and figures should not be interpreted in any way as limiting the scope of the present invention.

### Example

### Material & Methods

Analysis of human breast tumor samples by immunohistochemistry: One hundred twenty human primary breast tumor samples and three pairs of human primary breast carcinomas and their bone metastases were selected from the tumor bank of the Centre Leon Bérard (Lyon, France). Formalin-fixed, paraffin-embedded tumor tissues were used for immunohistochemical analysis. Four-micrometer-thick tissue sections were deparaffinized, rehydrated, and endogenous peroxidase activity was blocked in a sterile water solution containing 5% hydrogen peroxide. Tissue sections were incubated for 1 h at room temperature with rabbit polyclonal anti-ROBO1 antibody [1:200 dilution in an antibody diluent solution (Chemate, Dako, Trappes, France)] (Clark et al., 2002). Alternatively, the primary antibody was replaced by a non immune serum for negative control slides. After washing, tissue sections were incubated with a biotinylated secondary antibody bound to a streptavidine-peroxidase conjugate (LSAB+ kit, Dako, Trappes, France), and the signal developed with diaminobenzidine. Tissue sections were then counterstained with hematoxylin, dehydrated and mounted. The immunostaining intensity was evaluated independently by two investigators. The intensity of the staining was scored arbitrarily as follows: negative/weakly positive (+/−), moderately positive (2+), and strongly positive (3+). In case of disagreement between examiners, slides were reviewed and a consensus opinion obtained.

Cell lines and transfection: The MDA-MB-231/B02 human breast cancer cell line is a subpopulation of the MDA-MB-231 cancer line that was selected for the high efficiency with which it metastasizes to bone after i.v. inoculation (Peyruchaud et al., J Biol Chem., 278:45826-45832, 2003). A Flp-in-expressing subclone (MDA-MB-231/B02-Frt11), which has been previously generated (Buijs et al., Cancer Res., 67:8742-8751, 2007), was used here for cell transfection experiments. Human embryonic kidney (HEK) cells stably transfected to express human Slit-2 were obtained from Wu et al. (Nature, 410:948-952, 2001). Cells were routinely cultured in DMEM (Gibco) supplemented with 10% (v/v) heat-inactivated fetal calf serum (Perbio) and 1% penicillin/streptomycin (Gibco) at 37° C. in 5% CO₂.

The small hairpins RNA (siRNA) against human Robo1 were designed with the Promega Si Designer tool. The oligos used were as follows: ShRNA-RB1 forward, 5′-ACCgCAgTACTAAgggAA-CAATAAgTTCTCTATTgTTCCCTTAgTACTgCTTTTTC-3′(SEQ ID NO:1); ShRNA-RB1 reverse, 5′-TgCAgAAAAAgCAg-TACTAAgggAACAATAgAgAACTTATTgTTCCCTT-AgTACTg-3′ (SEQ ID NO:2).

The shRNAs and corresponding scrambles duplex were synthesized with the psiSTRIKE puromycin kit (Promega) under U6 promoter. MDA-MB-231/B02-Frt11 cells were transfected with the siRNA and scrambles duplex with Transfast Transfection Reagent (Promega), according to the manufacturer's instructions. Selection of the clones was obtained after growing the cells for two weeks in the presence of puromycin (2 μg/ml).

RNA isolation, reverse transcriptase-polymerase chain reaction (RT-PCR), and quantitative real-time PCR (qPCR): RNA was extracted from cells using the Nucleospin RNA II kit (Macherey-Nagel) and cDNA produced from 2 μg total RNA using the MMLV polymerase Promega. qPCR was performed using SYBR Green qPCR Kit (Finnzymes). Probes were purchased from Invitrogen.

Western blotting: Cells lysates were prepared from cells cultured in complete medium. Protein were electrophoresed on a 8% SDS-polyacrylamide gel and transferred onto a Immobilon transfer membrane (Millipore). Membranes were incubated with 5% (w/v) low fat-milk in PBS for 1 h at room temperature followed by 1 h incubation with a rabbit anti-Robo1 polyclonal antibody (diluted 1:50) (Clark et al., FEBS Lett., 523:12-16, 2002) or with a rabbit anti alpha-tubulin antibody (Sigma). Robo1 and alpha-tubulin were visualized using horseradish-peroxydase-donkey anti-rabbit IgG (Amersham) and enhanced chemiluminescence (Perkin Elmer).

Confocal microscopy: Subconfluent cell layers were fixed in 4% (w/v) formaldehyde for 30 min, washed three times with PBS and permeabilized for 5 min in PBS containing 0.2% (v/v) Triton-X-100. Permeabilized cells were then incubated with rabbit anti-Robo1 antibody ab7279 (17 μg/ml; AbCam) for 1 h, washed in PBS then incubated with a secondary, FITC-conjugated, anti-rabbit antibody (Sigma). Actin was labeled with phalloidin (Invitrogen) and nuclei were labeled with DAPI. Slides were viewed using confocal microscope Leica TCS-SP2.

Cell migration and invasion assays: Cell migration and invasion assays were performed in 24-well cell culture chambers with 8-μm diameter pore-size inserts (Becton Dickinson), as previously described (Zhang et al., Cancer Res., 67:5821-5830, 2007). Inserts were coated with 100-μl basement membrane Matrigel (3 μg/ml; Becton Dickinson) for 90 min at 37° C. in order to perform cell invasion experiments. Cancer cells (1.5×10⁵ cells/ml) were re-suspended in DMEM culture medium containing 0.1% (w/v) bovine serum albumin and 300-μl of this cell suspension were loaded into each insert (upper chamber). The chemoattractant [10% (v/v) fetal calf serum] was placed in the lower chamber (750 μl/well). The plates were incubated for 6 h at 37° C. in a 5% CO₂ incubator. After incubation, the inserts were collected carefully, the nonmigrating cells were removed and the migrating cells on the under surface of the inserts were fixed and stained with crystal violet. The membranes were mounted on glass slides and cells were counted under microscope.

The effects of Slit2, receptor factor kB ligand (RANKL) and osteoprotegerin (OPG) on cell migration were studied using 96-microwell plates (Millipore). Experiments were carried out essentially as described above with minor modifications. Briefly, inserts were loaded with 75 μl of a cell suspension (2×10⁵ cells/ml) and 150 μl of chemoattractant was placed in the lower chamber. The chemoattractant consisted of culture medium containing 0.5% (v/v) fetal calf serum with or without RANKL, OPG or the conditioned medium from Slit2-expressing HEK cells. Remaining of the experiments was as described above.

Scratch wound assay: Cells were seeded at a concentration of 1×10⁵ cells into 24-well plates and cultured for 24 hours at 37° C. in culture medium containing 10% (v/v) fetal calf serum. After incubation, the culture medium was removed and cells were further cultured in medium containing or not containing 5% (v/v) fetal calf serum, at which time a wound scratch was made on the cell monolayer by using a 20-μl micropipette tip. Cells were fixed, stained with crystal violet, and photographed 24 h later. Alternatively, cell migration was monitored by time-lapse videomicroscopy. Microscopic images were recorded, in a time lapse mode, with a CCD camera attached to a phase contrast microscope (Zeiss Axiovert 200M) and the behaviour of migrating cells was tracked over 24 hours using ImageJ software. Analysis of the images was performed using MetaMorph software.

Osteoclastogenesis assay: Experiments were conducted as described previously (Boucharaba et al., J Clin Invest., 114:1714-1725, 2004). Briefly, bone marrow cells from hind limbs of OF1 male mice were collected and seeded in 12-well tissue culture plates at a density of 1×10⁵ cells/ml per well in α-MEM medium (Invitrogen) supplemented with M-CSF (200 ng/ml) and RANK-L (150 ng/ml) (R&D Systems). Culture media were then supplemented with 10% (v/v) fetal calf serum in presence or absence of 2 μg/ml conditioned medium from transfectants. After 6 days, mature osteoclasts were enumerated under a microscope on the basis of the number of nuclei (more than three nuclei) and tartrate-resistant acid phosphatase (TRAP) activity (Sigma). Results were expressed as the number of osteoclasts per well.

Osteoblastogenesis assay: Cells were enzymatically isolated from the calvaria of 3-day-old OF-1 mice by sequential digestion with collagenase, as described previously (Bonnelye and Aubin, J Bone Miner Res., 17:1392-1400, 2002). Cells obtained from the last four of the five digestion steps (populations II-V) were plated in T-75 flasks in a-MEM containing 15% (v/v) heat-inactivated fetal calf serum (Sigma-Aldrich) and antibiotics including 100 μg/ml penicillin-streptomycine (Invitrogen) and 30 μg/ml fungizone (Invitrogen). After 24 h-incubation, attached cells were washed with PBS to remove non-viable cells and other debris, then collected by trypsinization using 0.02% trypsin-EDTA (Invitrogen). Aliquots were counted and the remaining cells re-suspended in the standard medium described above. The re-suspended cells were plated onto 24-well plates at 2×10⁴ cells/well. After 24 h incubation, the medium was changed and supplemented with 50 μg/ml ascorbic acid (Sigma-Aldrich). Ten millimolar sodium β-glycerophosphate (Sigma-Aldrich) was added for 1 week at the end of the culture. Medium was changed every 2 days. All dishes were incubated at 37° C. in a humidified atmosphere in a 95% air/5% CO₂ incubator. At the end of experiments, wells were fixed and stained with von Kossa, and bone nodules were counted on a grid. Results were plotted as the mean number of nodules ±SD of three wells for each experimental condition.

Cytokine array: A commercial antibody-based protein microarray designed to detect 79 growth factors, cytokines, and chemokines (RayBio Human Cytokine Array V, RayBiotech) was used. Experiments were carried out following manufacturer's instructions. Array membranes were blocked with the saturated buffer for 1 h and then incubated for 2 h with the conditioned medium (1 mL) from cultured transfected cells. After washing, membranes were incubated for 2 h with a cocktail of 79 biotinylated antibodies. Membranes were then washed and incubated for an additional 2 h with a peroxidase-labeled streptavidin solution. Detection of immunoreactive spots was carried out using an enhanced chemiluminescence detection system (GE Healthcare).

Animals: All procedures involving animals, including housing and care, method of euthanasia, and experimental protocols were conducted in accordance with a code of practice established by the local ethical committee (CREEA, Lyon, France). Studies were routinely inspected by the attending veterinarian to ensure continued compliance with the proposed protocols. Four-week-old female Balb/c homozygous (nu/nu) athymic mice were obtained from Charles River (St. Germain sur l'Arbresle, France).

Animal studies: Bone metastasis experiments were conducted in nude mice, as described previously (Boucharaba et al., J Clin Invest., 114:1714-1725, 2004; Peyruchaud et al., J Biol Chem., 278:45826-45832, 2003; Zhang et al., Cancer Res., 67:5821-5830, 2007; Buijs et al., Cancer Res., 67:8742-8751, 2007). MDA-MB-231/B02-Frt11 cells that had been stably transfected with shRNAs directed against Robo1 or Scrambles (5×10⁵ in 100 μL of PBS) were injected into the tail vein of nude mice anesthetized with 130 mg/kg ketamin and 8.8 mg/kg xylazin. Radiographs of anesthesized animals were taken weekly with the use of MIN-R2000 films (Kodak, Rochester, N.Y.) in an MX-20 cabinet X-ray system (Faxitron X-ray Corporation, Wheeling, Ill.). Osteolytic lesions were identified on radiographs as radio lucent lesions in the bone. The area of osteolytic lesions was measured using a Visio lab 2000 computerized image analysis system (Explora Nova, La Rochelle, France), and the extent of bone destruction per leg was expressed in square millimeters. Anesthetized mice were sacrificed by cervical dislocation after radiography on day 28.

Intraosseous tumor xenograft experiments were conducted in nude mice, as described previously (Zhang et al., 2007). Briefly, a small hole was drilled with a 30-gauge sterile needle through the tibial plateau with the knee flexed. Using a new sterile needle fitted to a 50-μl sterile Hamilton syringe (Hamilton Co., Reno, Nev.), a single-cell suspension (1×10⁵ cells in 30 μl of PBS) was injected in the bone marrow cavity. The progression of osteolytic lesions was monitored by radiography as described above. Anesthetized animals were sacrificed by cervical dislocation 7 weeks after tumor cell inoculation.

Bone histology and histomorphometry: Bone histology and histomorphometry analysis of bone lesions were performed as previously described (Boucharaba et al., 2000; Peyruchaud et al., 2003; Zhang et al., 2007). Following sacrifice of metastatic animals, both hind limbs from each animal were dissected, fixed in 80% (v/v) alcohol, dehydrated, and embedded in methylmethacrylate (MMA) or paraffin. A microtome (Microm, HM350S) was used to cut 7 to 9 μm-thick sections of undecalcified long bones, and the sections were stained with Goldner's trichrome. Histologic and histomorphometric analyses were performed on Goldner's trichrome-stained longitudinal medial sections of tibial metaphysis with the use of a computerized image analysis system (Visiolab 2000, Explora Nova, La Rochelle, France). Histomorphometric measurements (i.e., bone volume/tissue volume [BV/TV] and tumor burden/soft tissue volume [TB/STV] ratios) were performed in a standard zone of the tibial metaphysis, situated at 0.5 mm from the growth plate, including cortical and trabecular bone. The BV/TV ratio represents the percentage of bone tissue. The TV/STV ratio represents the percentage of tumor tissue.

The in situ detection of osteoclasts was performed on TRAP-stained longitudinal paraffin-embedded medial sections of tibial metaphysis with the use of a commercial kit (Sigma). Osteoclast resorption surface was calculated as the ratio of TRAP-positive trabecular bone surface to the total trabecular bone surface at the tumor-bone interface.

Statistical analysis. All data were analyzed with the use of StatView v5.0 software (version 5.0; SAS Institute Inc, Cary, N.C.). Data were analyzed with ANOVA followed by a Fisher's protected least significant difference (PLSD) test. Pairwise comparisons were carried out by performing nonparametric Mann-Whitney U test. P values less than 0.05 were considered statistically significant. All statistical tests were two-sided.

Results:

Expression of ROBO1 in human primary breast carcinomas and their bone metastases and in human breast cancer cells metastatic to bone in animals. As a first step towards evaluating ROBO1 in breast cancer bone metastasis, we did immunohistochemistry on 120 primary breast carcinomas using a rabbit polyclonal antibody directed against ROBO1. The scoring of ROBO1 staining intensity in tumors specimens showed that most of the tumors had a moderate or strong staining in tumor cells (61% and 31%, respectively), whereas only 8% of these tumors (10 out of 120) were negative or had a weak staining for ROBO1. Moreover, a higher ROBO1 expression in primary breast tumors was found to be associated with a higher risk of relapse in patients. In this respect, we did immunohistochemistry on three pairs of primary breast tumors and their matching bone metastases using an anti-ROBO1 polyclonal antibody. All of the matching primary breast and metastatic tumors expressed ROBO1, and the immunostaining was always homogenous (80 to 90% of the tumor cells were positive for ROBO1). The scoring of ROBO1 staining intensity in the three pairs of primary tumors and bone metastatic specimens showed a moderate-to-strong staining in tumor cells.

We previously selected a subpopulation of the MDA-MB-231 human breast cancer cell line (referred to as MDA-MB-231/B02) that only metastasizes to bone after i.v. inoculation (Peyruchaud et al., J Biol Chem., 278:45826-45832, 2003). Using A and B U133 Affymetrix GeneChips, we have compared the gene-expression profiles of MDA-MB-231 and MDA-MB-231/B02 cells and found that osteotropic MDA-MB-231/B02 cells express an osteoblast-like gene signature (Bellahcéne et al., Breast Cancer Res Treat., 101:135-148, 2007; Garcia et al., Clin Exp Metastasis, 25:33-42, 2008). Here, we compared gene expression levels of the ROBO/SLIT family between MDA-MB-231/B02 cells and parental MDA-MB-231 cells. From such a transcriptomic comparison, we observed that ROBO1 mRNA was overexpressed in MDA-MB-231/B02 cells. These results were confirmed by Western immunoblotting, showing a stronger ROBO1 protein expression in MDA-MB-231/B02 cells when compared to that observed with the parental cell line.

Contribution of ROBO1 in experimental breast cancer bone metastasis formation. Because immunohistochemical, transcriptomic and western immunoblotting data indicated a possible role of ROBO1 in the pathogenesis of breast cancer bone metastases, the expression of ROBO1 in human MDA-MB-231/B02 breast cancer cells was invalidated using siRNA strategy. Two transfectants (clones Sh1.32 and Sh1.33) were selected on the basis of their low expression of ROBO1 mRNA (real-time PCR) and protein (Western blotting), when compared to that observed in scrambled-transfected (clones SC2.1 and SC2.4) and MDA-MB-231/B02 parental cells. Quantitative real-time PCR analysis showed that ROBO1 transcript in clones Sh1.32 and Sh1.33 was down regulated by 90% when compared to that observed in scrambled-transfected SC2.1 and SC2.4 cells. ROBO1 silencing in human MDA-MB-231/B02 breast cancer cells did not affect ROBO4 mRNA expression levels. Moreover, confocal microscopic analysis of ROBO1 expression in scrambled-transfected cells clearly showed a strong immunostaining at the plasma membrane, which totally disappeared in ShRNA-transfected cells. In addition, scrambled-transfected cells seemed to attach firmly to the substrate, exhibiting robust actin cables running through the cytoplasm, whereas the actin cytoskeleton in ROBO1-deficient cells formed filopodial spikes at the plasma membrane, suggesting these cells were more motile.

MDA-MB-231/B02 transfectants were then injected into the tail vein of animals in order to examine the contribution of ROBO1 in the development of bone metastases in vivo. Radiographic analysis on day 25 after tumor cell inoculation revealed that mice bearing ROBO1-deficient tumors had osteolytic lesions that were 40% larger than those of mice bearing mock-transfected SC tumors. Similarly, histomorphometric analysis of hind limbs with metastases showed that animals bearing ROBO1-deficient tumors had statistically significantly lower BV/TV ratios (indicating a higher bone destruction) than mice bearing mock-transfected SC tumors. TRAP staining of bone tissue sections of metastatic legs showed that the active-osteoclast resorption surfaces at the tumor-bone interface did not however differ significantly between mice bearing ROBO1-deficient tumors or mock-transfected SC tumors. Compared with mice bearing SC tumors, there was also a 3-fold increase in the TB/STV ratio (a measure of the skeletal tumor burden) in legs from animals bearing ROBO1-deficient tumors.

Effects of ROBO1 silencing in breast cancer cells on differentiation of osteoclasts and osteoblasts. Bone-residing metastatic breast cancer cells secrete different cytokines (M-CSF, M-CP1) and interleukins (IL-6, IL-8 and IL-11) that stimulate the formation and activity of osteoclasts (Clezardin & Teti, Clin Exp Metastasis, 24:599-608, 2007). In addition, metastatic cells secrete factors (dickkopf-1, noggin) that inhibit osteoblast differentiation. To explore the possible cellular mechanisms responsible for the higher bone destruction in animals bearing ROBO1-deficient tumors, we examined whether conditioned medium from ROBO1-deficient cancer cells could modulate osteoclast and/or osteoblast formation.

We first used a human cytokine antibody array to measure 23 cytokines in the conditioned medium from mock-transfected MDA-MB-231/B02 cells (SC) and cells silenced for ROBO1 (Sh). Transfectants produced several cytokines and interleukins known to stimulate osteoclast activity. However, cytokine profiles were similar for SC- and Sh-transfected cells. Conditioned media used to measure human cytokine levels were then tested for their ability to stimulate osteoclastogenesis in vitro. Experiments were conducted in the presence of RANKL and M-CSF, which are two hematopoietic factors both necessary and sufficient to induce osteoclastogenesis (Clezardin & Teti, Clin Exp Metastasis, 24:599-608, 2007). The addition of the conditioned medium from SC- or Sh-transfected cells to M-CSF+RANKL increased osteoclast formation by 50%, when compared to that observed with M-CSF+RANKL alone (Ctrl). The osteoclast stimulatory activity of the conditioned medium from SC-transfected cells was not statistically different from that of Sh-transfected cells. As illustrated by the cytokine array, the osteoclast stimulatory activity of transfectants most likely depended on production of cytokines such as MCP-1, GM-CSF, IL-6 and IL-8. In addition, VEGF (which is another pro-osteoclastic factor) was found to be substantially produced by transfectants when compared to that observed with parental MDA-MB-231/B02 cells.

We next examined the ability of the conditioned media from Sh- and SC-transfected cells to modulate osteoblast differentiation and mineralization. Murine bone marrow-derived stromal cells differentiated into osteoblasts (as judged by alkaline phosphatase activity) and deposited calcium phosphate mineral (as judged by the von Kossa staining of bone nodules) when cultured in the presence of a mineralization medium consisting of sodium β-glycerophosphate and ascorbic acid. By contrast, the addition of conditioned media from Sh- and SC-transfected cells to the mineralization medium led to a 50% reduction in alkaline phosphatase activity and a near complete inhibition of bone nodule formation, when compared to that observed with the mineralization medium alone. The inhibitory activity of the conditioned medium from SC-transfected cells on osteoblast differentiation was not statistically different from that of Sh-transfected cells.

Taken together these results indicated that, independently of ROBO1 expression, MDA-MB-231/B02 cells stimulated osteoclast-mediated bone resorption and inhibited bone formation, explaining why bone destruction occurred in metastatic animals. The larger bone metastatic lesions in animals bearing ROBO1-deficient tumors were therefore not directly related to ROBO1 silencing but an indirect result of the greater number of ROBO1-deficient tumor cells residing in the bone marrow that stimulated osteoclast-mediated bone resorption. We next examined whether the larger bone metastatic lesions in animals bearing ROBO1-deficient tumors could result of a faster bone colonization by ROBO1-deficient cells.

Effects of ROBO1 silencing on breast cancer cell migration and invasion. We used several approaches to investigate whether ROBO1 could regulate the migration/invasion of breast cancer cells. Using a modified Boyden chamber assay, we first tested the effect of ROBO1 silencing on the chemotactic response of breast cancer cells to serum. There was a 1.5- to 2-fold increase in the migration of Sh-transfected cells (clones Sh1.32 and Sh 1.33) when compared to that observed with scrambled-transfected cells (clones SC2.1 and SC2.4 and parental B02 cells. Similarly, there was a substantial gain in invasion of Sh1.32 and Sh 1.33 cells when compared with scrambled-transfected and parental cells.

As an alternative experimental approach to the Boyden chamber assay, we tested the effect of ROBO1 silencing in a scratch wound assay. There was a statistically significant faster wound closure with Sh-transfected cells when compared to parental and scrambled-transfected cells. Similar results were obtained when Sh- and SC-transfected migrating cells were recorded by time-lapse videomicroscopy.

Thus these results suggested that ROBO1 silencing could draw breast cancer cells to faster colonize bone, thereby explaining the higher skeletal tumor burden in legs from animals bearing ROBO1-deficient tumors. This assumption was indeed supported by the observation that, when SC- and Sh-transfected cells were directly injected into the tibial bone marrow cavity, mice bearing transfected cells had a similar extent of bone destruction and skeletal tumor burden.

Expression of Slit2 in bone and role of Slit2 in the migration of breast cancer cells expressing ROBO1. It has been previously shown that Slit2 regulates osteoblast differentiation in vitro (Sun et al., Cell Tissue Organs, 190:69-80, 2009). Using reverse-transcription coupled to the polymerase chain reaction (RT-PCR), we found low Slit2 mRNA levels in the bone marrow from nude mice that did not receive human breast cancer cells. By contrast, there was a 100-fold increase in murine Slit2 mRNA levels in the bone marrow from metastatic animals. Slit2 was expressed by osteoblasts, but not osteoclasts and mononuclear cells as judged by RT-PCR. Moreover, Slit2 inhibited the migration of SC-transfected cells induced by FCS as opposed to that observed with Sh-transfected cells.

### REFERENCES

Throughout this application, various references describe the state of the art to which this invention pertains. The disclosures of these references are hereby incorporated by reference into the present disclosure.

1-15. (canceled)

16. A pharmaceutical composition for use in a method for the treatment of a bone mineral density related disease comprising
a ROBO1 modulator and
a pharmaceutically acceptable carrier.

17-18. (canceled)

19. A method of testing a patient thought to have or be predisposed to a bone density related disease, which comprises the step of analyzing a biological sample from said patient for one or both of:
a. detecting the presence of one or more of: a mutation in a gene encoding ROBO1, a mutation in a gene encoding SLIT2, and, an associated promoter of one or both of ROBO1 and SLIT2; and
b. analyzing the expression of one or both of a gene encoding ROBO1 and a gene encoding SLIT2.

20. The method of claim 19, wherein the presence of a mutation in the gene encoding ROBO1 and the gene encoding SLIT2 are detected in the detecting step.

21. The method of claim 19, wherein expression of a gene encoding ROBO1 and a gene encoding SLIT2 is analyzed in the analyzing step.

22. The method of claim 19, wherein said step of detecting is carried out using a technique selected from the group consisting of restriction fragment length polymorphism analysis, a hybridisation technique, DNA sequencing, exonuclease resistance, microsequencing, solid phase extension using ddNTPs, extension in solution using ddNTPs, an oligonucleotide assay, dynamic allele-specific hybridisation, ligation chain reaction, mini-sequencing, DNA “chips”, and allele-specific oligonucleotide hybridization.

23. The method of claim 19, wherein said step of analyzing is carried out by
nucleic acid amplification of DNA or mRNA transcripts encoding one or both of a ROBO1 and a SLIT2 gene; or
detecting one or both of a ROBO1 and a SLIT2 protein using an antibody, an antibody derivative or an antibody fragment which binds specifically to the protein translated from the gene encoding for ROBO1 or SLIT2.

24. A method for treating a bone mineral density related disease in a subject, comprising
administering to said subject a therapeutically effective amount of a ROBO1 modulator, a ROBO1 agonist or a ROBO1 antagonist.

25. The method of claim 24, wherein said ROBO1 modulator is administered, and said ROBO1 modulator is selected from the group consisting of small organic molecules, antibodies, aptamers and polypeptides.

26. The method of claim 24, wherein said bone mineral density related disease is selected from the group consisting of ghosal hematodiaphyseal dysplasia syndrome (GHDD), osteoporosis, osteoporosis associated with pseudoglioma, osteoporosis and oculocutaneous hypopigmentation syndrome, osteoporosis due to endocrinological dysfunction, osteogenesis imperfecta osteopenia, Paget's disease, osteomyelitis, hypercalcemia, osteonecrosis, hyperparathyroidism, lytic bone metastases, periodontitis, bone loss due to immobilization and osteoporosis associated with a disease selected from the group consisting of cachexia, anorexia, alopecia, rheumatoid arthritis, psoriatic arthritis, psoriasis, and inflammatory bowel disease.

27. The method of claim 24, wherein said ROBO1 agonist is administered, and said ROBO1 agonist is or comprises a SLIT2 polypeptide.

28. The method of claim 24, wherein said ROBO1 agonist is a fusion protein comprising the following segments:
a. a SLIT2 polypeptide and
b. an Fc domain of an immunoglobulin or a functionally-conservative variant thereof.

29. The method of claim 24, wherein said bone mineral density related disease is selected from the group consisting of osteoporosis, osteogenesis imperfecta osteopenia, Paget's disease, osteomyelitis, hypercalcemia, osteonecrosis, hyperparathyroidism, lytic bone metastases, and periodontitis.

30. The method of claim 24, wherein a ROBO1 antagonist is administered and said ROBO1 antagonist is selected from the group consisting of small organic molecules, antibodies, and aptamers.

31. The method of claim 30, wherein said antibodies and said aptamers are directed against one or both of ROBO1 and SLIT2.

32. The method of claim 24, wherein said bone mineral density related disease is Ghosal hematodiaphyseal dysplasia (GHDD) syndrome.

33. A method of treating a bone mineral density related disease associated with decreased bone mineral density in a subject, comprising
administering to said subject a therapeutically effective amount of i) a nucleic acid molecule encoding a SLIT2 polypeptide or a nucleic acid molecule encoding a fusion protein comprising a SLIT2 polypeptide; or ii) a therapeutically effective amount of an inhibitor of ROBO1 or SLIT2 gene expression.

34. The method of claim 33, wherein said nucleic acid molecule is present in a vector.

35. The method of claim 34, wherein said vector is selected from the group consisting of a plasmid, a cosmid, an episome, an artificial chromosome, a phage and a viral vector.

36. The method of claim 33, wherein said inhibitor of ROBO1 or SLIT2 gene expression is selected from the group consisting of anti-sense oligonucleotides, small inhibitory RNAs, short hairpin RNAs, and ribozymes.

37. A biomaterial or medical delivery device for local delivery of a ROBO1 modulator, comprising
a biocompatible material and
a ROBO1 modulator associated with said biocompatible material so as to allow local delivery of a therapeutically effective amount of the ROBO1 modulator.

38. The biomaterial or medical delivery device according to claim 37, wherein the biomaterial or medical delivery device is selected from the group consisting of a prosthesis, an internal patch around bone, and a bone implant.
